(19) United States
(12) Patent Application Publication
(10) Pub. No.: US 2021/0137828 A1
(43) Pub. Date: May 13, 2021

(54) Cannabidiol Sublingual Spray Formulations

(71) Applicant: Horng-Shin Jen Li, Chandler, AZ (US)

(72) Inventor: Horng-Shin Jen Li, Chandler, AZ (US)

(21) Appl. No.: 16/681,361

(22) Filed: Nov. 12, 2019

(51) Int. Cl. A61K 9/00; A61K 31/05; A61K 9/08; A61K 47/10; A61K 47/26

(52) U.S. Cl. CPC A61K 9/006; A61K 31/05; A61K 47/26; A61K 47/10; A61K 9/08

(57) ABSTRACT

Sublingual cannabidiol formulations that are stable at room or refrigerated temperatures. It may also possess improved absorption, faster onset, increased bioavailability and preferred flavor. The dose range from 12.5 mg to 25 mg per spray.

### CROSS REFERENCE TO RELATED APPLICATIONS

This patent application claims priority of U.S. Provisional Application No. 62/785,624 filed on Dec. 27, 2018.

### FIELD OF INVENTION

The present invention specifies a lipid-based cannabidiol sublingual spray solution comprising cannabidiol, a lipid solvent, a sweetener, a flavoring agent, ethanol, and an antioxidant. The formulation is stable at room or refrigerated temperatures and has an improved absorption, bioavailability and taste compare to current cannabidiol oral sprays.

### BACKGROUND OF THE INVENTION

The increasing use of cannabidiol in medicine has led to finding more effective ways of drug delivery. This is part due to factors, such as, poor aqueous solubility, limited bioavailability, and cannabidiol instability. Despite various cannabidiol products on the market to date, an optimal sublingual spray that is stable with faster onset, increased absorption, improved bioavailability, and favorable taste has not been achieved. In the pharmaceutical industry, there is currently Epidiolex on the market that is formulated in sesame seed oil, sucralose, strawberry flavoring and up to 10% v/v ethanol. While in the nutraceutical world, examples include Green Earth Medicinals Sublingual Spray and CBD Luxe Organic CBD Sublingual/Topical Spray that are not optimal from a consumer stand point.

### SUMMARY OF THE INVENTION

The present invention is directed to a sublingual spray, and more particularly to a lipid based cannabidiol (naturally extracted) formulation comprising MCT oil, sucralose, flavoring agent, ethanol and an antioxidant. All percentages of ingredients reported herein are expressed as weight by weight, unless otherwise indicated. It is a further object of the invention to provide formulations comprising cannabidiol (25mg per spray or less) and the following ingredients: (i) less than 10% ethanol (ii) less than 0.4% sucralose (iii) less than 0.4% flavoring agent such as grape or strawberry, (iv) less than 0.4% CoQ10, (v) and total to 100% with MCT oil wherein the formulations are suitable for sublingual administration. In one embodiment, the provided formulation contains up to 10% ethanol in the absence of anti-oxidant. In another embodiment, the percentage of flavoring agent was 0.2% or 0.4%. In all embodiments, the combined total of all ingredients is 100%.

Stability data was established for formulations with 25 mg/spray, 0.2% sucralose, 0.4% strawberry flavor, and 10% ethanol in the presence of MCT oil. The results indicated that assay and impurity levels were within USP and ICH specifications at the accelerated condition of 40C at the one month time point. This can be extrapolated to assigning a one year expiration at room temperature for lipid based formulation. See Table 1 through 2 for stability data of impurities and assay.

TABLE 1
Impurity Stability Data for CBD Sublingual Spray Formulation

| | | | | | | T = 4wk | |
|---|---|---|---|---|---|---|---|
| Impurities | RT | RRT | T = 0 | T = 2wk 25 C. | T = 2wk 40 C. | 25 C. | T = 4wk 40 C. |
| Unknown | 4.341 | 0.40 | 0.04 | 0.04 | 0.05 | 0.04 | ND |
| Unknown | 4.539 | 0.41 | ND | 0.04 | 0.10 | 0.09 | 0.19 |
| Unknown | 4.97 | 0.45 | ND | 0.08 | 0.21 | 0.17 | 0.42 |
| Unknown | 6.418 | 0.59 | 0.50 | 0.50 | 0.50 | 0.50 | 0.49 |
| Unknown | 8.237 | 0.75 | 0.23 | 0.23 | 0.25 | 0.23 | 0.26 |
| Unknown | 8.591 | 0.78 | ND | ND | ND | ND | 0.06 |
| Unknown | 10.44 | 0.95 | ND | ND | 0.21 | 0.15 | 0.43 |
| CBD | 10.945 | 1.00 | 99.24 | 99.05 | 98.60 | 98.75 | 97.96 |
| Unknown | 14.107 | 1.29 | ND | ND | ND | ND | 0.05 |
| Unknown | 20.047 | 1.83 | ND | 0.05 | 0.09 | 0.08 | 0.14 |

TABLE 2
Assay Stability Data for CBD Sublingual Spray Formulation

| | | T = 2 wk | T = 2 wk | T = 4 wk | T = 4 wk |
|---|---|---|---|---|---|
| Sample | T = 0 | 25 C. | 40 C. | 25 C. | 40 C. |
| 81318 | 100 | 105.07 | 104.67 | 99.44 | 98.00 |

### DETAILED DESCRIPTION

Lipophilic compounds such as cannabidiol (CBD) that are unstable in the presence of oxygen and moisture have proven difficult to formulate into stable formulations due to degradation and insolubility. However there has been an increasing demand for a more optimal formulation of CBD due to its numerous health benefits. The current invention allows ease of administration with an increased absorption, bioavailability, stability, and optimal taste. The improved absorption is mainly due to the presence of ethanol at 5% (w/w) which facilitates the absorption of CBD across the sublingual cell membrane. The lower ethanol formulations are more desirable for younger children. With the presence of a lipid such as MCT oil, the CBD is protected during the absorption process into the blood stream and eventually to the receptor sites in the endocannabinoid system. Thus the bioavailability is greatly improved. As far as stability, the addition of anti-oxidants which includes CoQ10 helps stabilize the formulation against oxidation and increases shelf-life. The amount of antioxidants in current invention is 0.2%. Finally, with the presence of a sweetener such as sucralose the taste is more favored compare to the currently available products that are also lipid based formulations where the taste needs masking. Sweetener levels will be similar to the level of antioxidant. See Tables 2 through 7 for formulations CBD sublingual spray evaluated.

TABLE 2
25 mg/spray with 10% Ethanol

| Component | Percent w/w |
|---|---|
| Ethanol | 10 |
| CBD | 8.33 |
| Sucralose | 0.2 |
| Flavor | 0.4 |
| MCT Oil | q.s. to 100% |

TABLE 3
12.5 mg/spray with 10% Ethanol

| Component | Percent w/w |
|---|---|
| Ethanol | 10 |
| CBD | 4.17 |
| Sucralose | 0.2 |
| Flavor | 0.4 |
| MCT Oil | q.s. to 100% |

TABLE 4
25 mg/spray with 5% Ethanol

| Component | Percent w/w |
|---|---|
| Ethanol | 5 |
| CBD | 8.33 |
| Sucralose | 0.2 |
| Flavor | 0.4 |
| MCT Oil | q.s. to 100% |

TABLE 5
12.5 mg/spray with 5% Ethanol

| Component | Percent w/w |
|---|---|
| Ethanol | 5 |
| CBD | 4.17 |
| Sucralose | 0.2 |
| Flavor | 0.4 |
| MCT Oil | q.s. to 100% |

TABLE 6
25 mg/spray with 5% Ethanol

| Component | Percent w/w |
|---|---|
| Ethanol | 5 |
| CBD | 8.33 |
| Sucralose | 0.2 |
| CoQ10 | 0.2 |
| Flavor | 0.4 |
| MCT Oil | q.s. to 100% |

TABLE 7
12.5 mg/spray with 5% Ethanol

| Component | Percent w/w |
|---|---|
| Ethanol | 5 |
| CBD | 4.17 |
| Sucralose | 0.2 |
| CoQ10 | 0.2 |
| Flavor | 0.4 |
| MCT Oil | q.s. to 100% |

The formulation of the present invention may offer the benefits of CBD described herein. Among common benefits, natural pain relief tops the list for many. Evidence suggests that cannabinoids may prove useful in pain modulation by inhibiting neuronal transmission in pain pathways. A 2012 study published in the Journal of Experimental Medicine found that CBD significantly suppressed chronic inflammatory and neuropathic pain in rodents without causing analgesic tolerance. Researchers suggest that CBD and other non-psychoactive components of CBD may represent a novel class of therapeutic agents for the treatment of chronic pain. (J Exp Med. June 4; 209(6):1121-34) According to a 2007 meta-analysis conducted in Canada, the use of CBD buccal spray was found to be effective in treating neuropathic pain in multiple sclerosis, which can be debilitating for 50 to 70 percent of MS patients. (Curr Med Res Opin. 2007 January; 23(1):17-24)

Research shows that CBD other benefits include producing antipsychotic effects. It appears to have a pharmacological profile like antipsychotic drugs as seen using behavioral and neurochemical techniques in animal studies. Additionally, studies show that CBD prevents human experimental psychosis and is effective in open case reports and clinical trials in patients with schizophrenia, with a remarkable safety profile. (Curr Pharm Des. 2012; 18(32):5131-40) Also studies using animal models of anxiety and involving healthy volunteers clearly suggest an anxiolytic-like effect of CBD. Cannabidiol has shown to reduce anxiety in patients with social anxiety disorder and researchers suggest that it may also be effective for panic disorder, obsessive compulsive disorder, and post-traumatic stress disorder. (Rev Bras Psiquiatr. 2012 June; 34 Suppl 1:S104-10)

In addition, several scientific reports demonstrate that CBD benefits include possessing antiproliferative, pro-apoptotic effects that inhibit cancer cell migration, adhesion and invasion. (Br J Clin Pharmacol. 2013 February; 75(2): 303-312) A 2006 study published in the Journal of Pharmacology and Experimental Therapeutics found for the first time that CBD potently and selectively inhibited the growth of different breast tumor cell lines and exhibited significantly less potency in non-cancer cells. (J Pharmacol Exp Ther. 2006 September; 318(3):1375-87) In 2011, researchers added light on the cellular mechanism through which CBD induces cell death in breast cancer cells. They showed that CBD induced a concentration-dependent cell death of both estrogen receptor-positive and estrogen receptor-negative breast cancer cells. They also found that the effective concentrations of CBD in tumor cells have little effect on non-tumorigenic, mammary cells. (Mol Cancer Ther. 2011 July; 10(7):1161-72) CBD behaves as a non-toxic compound and studies show that doses of 700 milligrams per day for 6 weeks did not show any overt toxicity in humans, suggesting that it can be used for prolonged treatment. Not only does the research show that CBD benefits including being effective in fighting breast cancer cells, data also suggests that it can be used to inhibit the invasion of lung and colon cancer, plus it possesses anti-tumor properties in gliomas and has been used to treat leukemia. (Br J Clin Pharmacol. 2013 February; 75(2): 303-312) A 2012 study published in the British Journal of Pharmacology found that CBD benefits including possessing anti-nausea and antiemetic effects when it was administered. (Br J Pharmacol. 2012 April; 165(8):2620-34) Researchers found that CBD acts in a diphasic manner, meaning that in low doses it suppresses toxin-induced vomiting.

Other benefits also include a 2014 survey conducted by researchers at Stanford University on the use of cannabidiol to treat their child's seizures. Nineteen responses met the inclusion criteria for the study: a diagnosis of epilepsy and current use of CBD. The average number of anti-epileptic drugs tried before using CBD was 12. Sixteen (84 percent) of the 19 parents reported a reduction in their child's seizure frequency while taking CBD. Of these, two (11 percent) reported complete seizure freedom, eight (42 percent) reported a greater than 80 percent reduction in seizure frequency, and six (32 percent) reported a 25-60 percent seizure reduction. Other beneficial effects included increased alertness, better mood and improved sleep; while side effects included drowsiness and fatigue. (Epilepsy Behav. Author manuscript; available in PMC 2014 Dec. 1) Later in 2014, researchers reported on preliminary results of a study involving children with treatment-resistant epilepsies in an expanded access “compassionate use program.” Patients received a purified 98 percent oil-based CBD extract called Epidiolex, which is made by GW Pharmaceuticals. After 3 months of treatment, 39 percent of the 23 patients had more than a 50 percent reduction in seizures, with a 32 percent median reduction. These preliminary results support the animal studies and survey reports that CBD may be a promising treatment for treatment-resistant epilepsy and it is generally well-tolerated in doses up to 25 milligrams per kilogram of body weight. (Orrin Devinsky, (Abst. 3.303), 2014)

Lastly a 2006 study that found CBD treatment significantly reduced the incidence of diabetes in non-obese diabetic mice from an incidence of 86 percent in non-treated mice to an incidence of 30 percent in CBD-treated mice. CBD benefits also showed a significant reduction of plasma levels of pro-inflammatory cytokines. A histological examination of the pancreatic islets of the CBD-treated mice revealed significantly reduced insulitis. (Autoimmunity. 2006 March; 39(2):143-51) A 2013 study published in the British Journal of Clinical Pharmacology reports that CBD protects against the vascular damage caused by a high glucose environment, inflammation or the induction of type 2 diabetes in animal models; plus, CBD proved to reduce the vascular hyperpermeability (which causes leaky gut) associated with such environments. (Br. J Clin Pharmacol. 2013 February; 75(2):313-22)

Besides the inherent benefits of cannabidiol itself in the current invention, it also provides ease of administration through the sublingual route. The oral cavity offers a simple, painless method of cannabidiol administration. Within the oral cavity, there are three generally recognized routes of administration of an active agent, namely local, buccal and Sublingual. Local delivery is mainly limited to applications regarding disruptions occurring within the oral cavity itself such as a canker sore. The buccal mucosa area encompasses the mucosal membranes of the inner lining of the cheeks. The buccal mucosa is however, less permeable than the sublingual area. One of the major disadvantages associated with buccal mucosa delivery of an active agent has been the relatively low passage of active agents across the mucosal epithelium, thereby resulting in low agent bioavailability, which translates into a substantial loss of usable active agent within each dosage. Sublingual delivery is achieved through the mucosal membranes lining the floor of the mouth. Because of the high permeability and the rich blood supply, transport via the sublingual route results in rapid in vivo absorption. Sublingual delivery is also beneficial in providing a delivery route appropriate for highly permeable drugs with short delivery period requirements and an infrequent dosing regimen. The sublingual administration of cannabidiol is advantageous over other forms of administration in that it does not require injection using a syringe and needle, and avoids the need for formulating unit dose oral formulations. Preferably the sublingual administration of cannabidiol in accordance with the present invention is suitable for ease of self-administration along with its other benefits.

What is claimed is:

1. A cannabidiol containing sublingual solution comprises: cannabidiol, a lipid solvent, a sweetener, anti-oxidant and ethanol, characterized in that the sweetener is an ultrahigh potency sweetener.

2. A cannabidiol containing sublingual solution as claimed in any of the preceding claims which further comprises a flavoring agent.

3. A cannabidiol containing sublingual solution as claimed in any of the preceding claims wherein the cannabidiol is present in an amount of from 12.5 to 25 mg per spray, ethanol is present in an amount of less than 10% (w/w), ultrahigh potency sweetener is present in less than 0.2% (w/w), flavoring agent is present in an amount of less 0.4% (w/w) and lipid solvent is q.s. to 100%.

4. A cannabidiol containing sublingual solution as claimed in claim 7, wherein the ultrahigh potency sweetener is sucralose, and the lipid solvent is MCT oil.

5. A cannabidiol containing sublingual solution as claimed in any of the preceding claims which is stable in climatic zones I and II for up to 12 months at 25° C.

6. A cannabidiol containing oral solution as claimed in any of the preceding claims which is stable in climatic zones Ill and IV for up to 1 months at 40° C.

7. A cannabidiol containing sublingual solution as claimed in any of the preceding claims which is contains a stabilizing agent.
